(19) United States
(12) Patent Application Publication
(10) Pub. No.: US 2025/0249234 A1
(43) Pub. Date: Aug. 7, 2025

(54) Therapy Applicator Head

(71) Applicant: SHENZHEN NUON MEDICAL EQUIPMENT CO., LTD., Shenzhen (CN)

(72) Inventors: ALAIN DIJKSTRA, Amstelveen (NL); LI XIANG, Shenzhen (CN)

(73) Assignee: SHENZHEN NUON MEDICAL EQUIPMENT CO., LTD., Shenzhen (CN)

(21) Appl. No.: 18/602,115

(22) Filed: Mar. 12, 2024

(30) Foreign Application Priority Data
Feb. 4, 2024 (CN) 202420269411X
Feb. 4, 2024 (CN) 2024202799323

(51) Int. Cl. A61N 1/04; A61L 2/10; A61L 2/26; A61M 35/00; A61N 5/06; H02J 7/00

(52) U.S. Cl. CPC A61N 1/0472; A61L 2/10; A61L 2/26; A61M 35/00; A61N 5/0616; H02J 7/0042; A61L 2202/11; A61L 2202/24; A61N 2005/0626; A61N 2005/0659; A61N 2005/0661; A61N 2005/0663

(57) ABSTRACT

Embodiments of the present invention provide a therapy applicator head. The therapy applicator head includes a housing container including an applicator body. The therapy applicator head further includes an applicator plate provided in an upper portion of the applicator body. The applicator plate is inclined at a predetermined angle. Furthermore, the therapy applicator head includes one or more stimulation elements located underneath the applicator plate. The therapy applicator head also includes a rechargeable electrical power source located below the applicator plate. The rechargeable electrical power source configured to supply electrical power to the one or more stimulation elements. Furthermore, the applicator plate further includes a dispensing hole located in an uphill portion on an upper surface of the applicator plate, the dispensing hole configured to be fluidically connected to a serum container through a fluidic channel.

### TECHNICAL FIELD

The present invention relates generally to devices used in the application of therapy on external surfaces of the body of a user. More specifically, the present invention relates to therapeutic applicator heads that are used in the application of therapies such as Photo-Biomodulation, electrode therapy, ultrasonic wave therapy, heating, cooling, and application of therapeutic serums.

### BACKGROUND ART

The human skin, the largest organ of the body, serves as a protective barrier against infections, dehydration, and harmful radiation like ultraviolet light. It also produces Vitamin D when exposed to sunlight, making skincare essential for overall health. External factors and internal conditions can lead to skin disorders and various conditions such as wrinkles, lesions, and acne. Given that skin conditions negatively contribute to aesthetic appeal, people often opt for non-invasive or minimally invasive treatments and cosmetic products to maintain skin health. These treatments and products are typically applied using therapy applicators.

Serum applicators, also known as velvet applicators within the cosmetics industry, are available in various shapes and sizes to suit different purposes. For instance, mitt-shaped self-tanning applicators ensure a smooth and streak-free application of self-tanner products. Makeup applicators are used for applying foundation, blush, and bronzer, while lipstick applicators are tailored for precise lipstick application. In pharmaceutical settings, applicators take different forms such as swabs, sponge sticks, and pre-saturated pads for applying antiseptic solutions to treat skin infections and dress wounds. Applicator bottles containing reservoirs of antiseptic solutions, along with applicator tips or brushes, are also utilized for antiseptic purposes.

As evident from the preceding discussion, various types of serums and applications require different constructions of applicators. Few existing solutions have the versatility to serve multiple purposes. Consequently, users may find themselves needing to acquire numerous devices mentioned earlier to address diverse situations, resulting in elevated costs and material wastage.

Therefore, there is a need in the art for a therapy applicator head that does not suffer from the aforementioned deficiencies.

### OBJECTS OF THE INVENTION

Some of the objects of the present invention are as follows:

An object of the present invention is to provide a therapy applicator head that can be used for both cosmetics and medicinal purposes.

Another object of the present invention is to provide a therapy applicator head that is capable of attaching to several different reservoirs containing different kinds of serums and applying those serums to the external surfaces of the body of a user.

Another object of the present invention is to provide a therapy applicator head that can provide other kinds of therapies such as irradiation, ultrasonic wave therapy, vibratory therapy, electrode therapy, and the like, in addition to the serum application.

Another object of the present invention is to provide a therapy applicator head that is simple in construction and convenient for assembly, cleaning, servicing, reusing, and recycling.

Another object of the present invention is to provide a therapy applicator head that can be used for different forms of substances such as facial serums, skin moisturizers, oils, and pharmaceutical compositions, etc. that may be available in the form of liquids, gels, emulsions, aerosols, and solutions, etc. and are used for treating skin-related conditions and/or enhancing the overall aesthetic appeal and general health of the skin of a user.

### SUMMARY OF THE INVENTION

According to a first aspect of the present invention, there is provided a therapy applicator head. The therapy applicator head includes a housing container. The housing container includes an applicator body. Furthermore, the therapy applicator head includes an applicator plate provided in an upper portion of the applicator body. The applicator plate is inclined at a predetermined angle. The therapy applicator head further includes one or more stimulation elements located underneath the applicator plate. The therapy applicator head also includes a rechargeable electrical power source located below the applicator plate. The rechargeable electrical power source is configured to supply electrical power to the one or more stimulation elements. Furthermore, the applicator plate further includes a dispensing hole located in an uphill portion on an upper surface of the applicator plate. The dispensing hole is configured to be fluidically connected to a serum container through a fluidic channel. Also, in use, a serum contained in the serum container is configured to flow downhill from the dispensing hole and along the inclined applicator plate.

In one embodiment of the invention, the applicator plate is made from a thermally conducting material and/or an electrically conducting material.

In one embodiment of the invention, the predetermined angle of the applicator plate is adjustable.

In one embodiment of the invention, the therapy applicator head further includes at least two charging terminals provided on the upper surface of the applicator plate, the at least two charging terminals configured to receive electrical power to charge the rechargeable electrical power source.

In one embodiment of the invention, the therapy applicator head further includes a removable cap configured to be fastened onto the housing container. The removable cap includes enclosed therewithin at least two power terminals configured to make contact with the at least two respective charging terminals, to supply electrical power to the at least two charging terminals from an external power source.

In one embodiment of the invention, the removable cap and the one or more stimulation elements comprise an Ultraviolet (UV) light source configured to sterilize the upper surface of the applicator plate.

In one embodiment of the invention, the housing container includes a first engaging arrangement configured to engage with a second engaging arrangement of the serum container, the second engaging arrangement complementary to the first engaging arrangement, to fasten the housing container to the serum container.

In one embodiment of the invention, the one or more stimulation elements are selected from a group consisting of irradiation sources, heating elements, cooling elements, vibratory elements, ultrasonic wave generators, electrodes, and combinations thereof.

In one embodiment of the invention, the irradiation sources are configured to emit electromagnetic radiation in wavelengths ranging between 300 nm and 1200 nm.

In one embodiment of the invention, the therapy applicator head further includes a user interface configured to receive control input signals to modify operational characteristics of the one or more stimulation elements.

In one embodiment of the invention, the therapy applicator head further includes a processor, a memory unit operably connected with a processor, and a pressure sensor mechanically coupled to the applicator plate. The memory unit comprises machine-readable instructions, the machine-readable instructions when executed by the processor, enable the processor to receive a pressure input from the pressure sensor, indicative of a predetermined amount of pressure applied onto the applicator plate, and allow the serum to be dispensed from the dispensing hole in response to sensing the predetermined amount of pressure.

In one embodiment of the invention, the processor is further enabled to operate an electronically controlled valve to allow the serum to be dispensed from the dispensing hole.

In one embodiment of the invention, the processor is further enabled to operate an electronically controlled pump to allow the serum to be dispensed from the dispensing hole.

In one embodiment of the invention, the therapy applicator head further includes a skin condition sensor mechanically coupled to the applicator plate. Furthermore, the processor is further enabled to receive skin condition data from the skin condition sensor, and in correlation with the skin condition data, perform one or more of modifying operational characteristics of the one or more stimulation elements, and allowing a predetermined amount of serum to be dispensed from the dispensing hole.

According to a second aspect of the present invention, there is provided a therapy applicator head. The therapy applicator head includes a housing container including an applicator body. Furthermore, the therapy applicator head includes an applicator plate provided in an upper portion of the applicator body. The therapy applicator head further includes one or more stimulation elements located underneath the applicator plate. Furthermore, the therapy applicator head includes a rechargeable electrical power source located below the applicator plate, the rechargeable electrical power source configured to supply electrical power to the one or more stimulation elements. Furthermore, the therapy applicator head includes at least two charging terminals provided on an upper surface of the applicator plate, the at least two charging terminals configured to receive electrical power to charge the rechargeable electrical power source. The therapy applicator head also includes a removable cap configured to be fastened onto the housing container. Furthermore, the removable cap includes enclosed therewithin at least two power terminals configured to make contact with the at least two respective charging terminals provided on the upper surface of the applicator plate, to supply electrical power to the at least two charging terminals from an external power source.

In one embodiment of the invention, the applicator plate inclined at a predetermined angle, the predetermined angle of the applicator plate being adjustable.

In one embodiment of the invention, the removable cap and the one or more stimulation elements include an Ultraviolet (UV) light source configured to sterilize the upper surface of the applicator plate.

According to a third aspect of the present invention, there is provided a method of using a therapy applicator head. The method includes providing the therapy applicator head. The therapy applicator head includes a housing container. The housing container includes an applicator body. Furthermore, the therapy applicator head includes an applicator plate provided in an upper portion of the applicator body, the applicator plate inclined at a predetermined angle. The therapy applicator head further includes one or more stimulation elements located underneath the applicator plate. Furthermore, the therapy applicator head includes a rechargeable electrical power source located below the applicator plate, the rechargeable electrical power source configured to supply electrical power to at least the stimulation element. The therapy applicator head also includes at least two charging terminals provided on an upper surface of the applicator plate, the at least two charging terminals configured to receive electrical power to charge the rechargeable electrical power source. The method further includes fastening the housing container to a serum container, the housing container including a first engaging arrangement configured to engage with a second engaging arrangement of the serum container, the second engaging arrangement complementary to the first engaging arrangement. Furthermore, the applicator plate further includes a dispensing hole on the upper surface of the applicator plate, the dispensing hole fluidically connected to the serum container through a fluidic channel. Furthermore, the method includes applying the applicator plate to a body portion of a user, as a result wherein, a serum stored in the serum container is applied, to the body portion of the user, through the fluidic channel. Also, the method includes activating the one or more stimulation elements through a user interface configured to receive control input signals to modify operational characteristics of the one or more stimulation elements.

In one embodiment of the invention, the method further includes fastening a removable cap onto the housing container, as a result wherein, at least two power terminals, enclosed within the removable cap, make contact with the at least two respective charging terminals provided on the upper surface of the applicator plate, to supply electrical power to the at least two charging terminals from an external power source.

In one embodiment of the invention, the method further includes sterilizing the upper surface of the applicator plate through an Ultraviolet (UV) light source included within the removable cap and the one or more stimulation elements.

In the context of the specification, the terms “polymer” or “plastic” refer to a material made up of long chains of organic molecules (having eight or more organic molecules) including, but not limited to, carbon, nitrogen, oxygen, and hydrogen as their constituent elements. The term polymer is envisaged to include both naturally occurring polymers such as wool, and synthetic polymers such as polyethylene and nylon.

In the context of this specification, the term “serum” includes, within its scope, facial serums, skin moisturizers, oils, and pharmaceutical compositions, etc. that may be available in form of pure substances, colloids, liquids, gels, emulsions, aerosols, and solutions, etc. and are used for treating skin-related conditions and/or enhancing overall aesthetic and general health of the skin of a user.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1A illustrates a perspective view of an assembly of a therapy applicator head and a serum container, in accordance with an embodiment of the present invention;

FIG. 1B illustrates an exploded view of the assembly of FIG. 1A;

FIG. 2A illustrates an exploded view of a removable cap of the therapy applicator head, in accordance with an embodiment of the present invention;

FIG. 2B illustrates a sectional view of the removable cap, in accordance with an embodiment of the present invention;

FIG. 3A illustrates an exploded view depicting a housing container of the therapy applicator head and elements contained within the housing container, in accordance with an embodiment of the present invention;

FIG. 3B illustrates an applicator plate and a fluidic channel coupled to the applicator plate, in accordance with an embodiment of the present invention;

FIG. 4A illustrates a sectional view of the housing container and the elements contained therewithin, in accordance with an embodiment of the present invention;

FIG. 4B illustrates a sectional view of the housing container and the elements contained therewithin, in accordance with another embodiment of the present invention;

FIG. 5 illustrates a method of using the therapy applicator head, in accordance with an embodiment of the present invention;

FIG. 6 illustrates an application scenario depicting the assembly of the therapy applicator head and the serum container being applied to a facial region of a user, in accordance with an embodiment of the present invention; and

FIG. 7 illustrates an application scenario depicting the therapy applicator head in communication with a user computing device, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

Embodiments of the present invention provide a therapy applicator head. The therapy applicator head includes a housing container that is configured to be fastened to a serum container. The housing container includes an applicator plate in an upper portion of the housing container. The applicator plate may be made up of a thermally and/or electrically conducting material. The applicator plate is inclined at a predetermined angle and includes a dispensing hole in an uphill portion of an upper surface of the applicator plate. The dispensing hole is configured to be fluidically connected to the serum container through a fluidic channel. Therefore, in use, a serum contained in the serum container is configured to flow downhill from the dispensing hole and along the inclined applicator plate. The applicator plate is coupled to an applicator body which in turn is coupled to the housing container through a fastening arrangement such as snap rings. In several embodiments of the invention, the applicator body includes a mechanism to change the predetermined angle of inclination of the applicator plate.

Furthermore, the applicator plate is provided with charging terminals for charging one or more rechargeable batteries provided within the housing container. The one or more rechargeable batteries are configured to provide electrical power to one or more stimulation elements located underneath the applicator plate. The one or more stimulation elements, in that regard, may be selected from a group consisting of irradiation sources, heating elements, cooling elements, ultrasonic wave generators, electrodes, vibratory elements, and the like. In that regard, the one or more stimulation elements would provide additional stimulation therapy in addition to the serum application through the dispensing hole. In several embodiments, a pressure sensor may be provided under the applicator plate that may sense a pressure being applied onto the upper surface of the applicator plate. A processor executing machine-readable instructions may receive pressure signals from the pressure sensor and may activate the one or more stimulation elements and allow the serum to be dispensed through the dispensing hole on sensing a predetermined amount of threshold pressure applied onto the applicator plate. In that regard, the processor may operate an electronically controlled valve or an electronically controller pump to allow the serum to be dispensed.

The applicator plate may also be provided with several sensors, such as a skin condition sensor may be provided flush with the upper surface of the applicator plate. The skin condition sensor may be configured to sense the condition of the skin of the user. In that regard, the skin condition sensor may be optical or an infrared sensor sensing taking images of surface abnormalities in the skin. The skin condition sensor may also be a contact-type displacement sensor or an accelerometer that may generate signals in correlation with abrasion patterns, acne, or bumps on the surface of the skin. In that regard, the processor may modify the operational characteristics of the one or more stimulation elements and the electronically controlled valve and/or the electronically controlled pump in correlation with the signals received from the skin condition sensor. In several embodiments of the invention, the therapy applicator head may also include a removable cap with power terminals enclosed therewith. The power terminals would make contact with the charging terminals of the applicator plate to supply electrical power for charging the one or more rechargeable batteries contained within the housing container, from an external power source. The removable cap may also include an Ultraviolet (UV) light source configured to sterilize the upper surface of the applicator plate. However, in several embodiments, the one or more stimulation elements including irradiation sources configured to generate the UV light may also be deployed to sterilize the applicator plate.

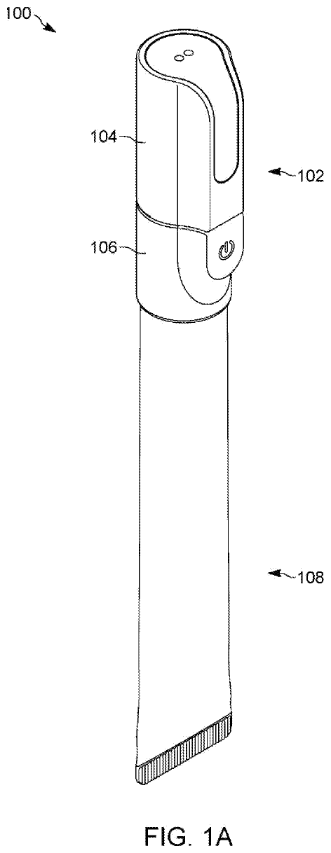

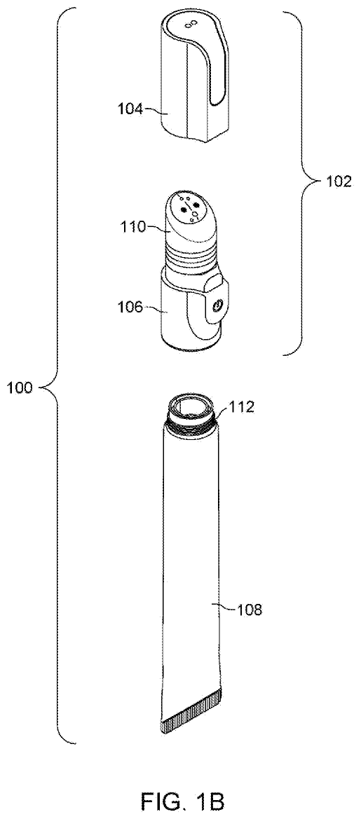

Several embodiments of the present invention will now be discussed in detail with reference to FIGS. 1A-7.

FIG. 1A illustrates a perspective view of an assembly 100 of a therapy applicator head 102 (hereinafter also referred to as “the head 102”) and a serum container 108, in accordance with an embodiment of the present invention. FIG. 1B illustrates an exploded view of the assembly 100 of FIG. 1A. The head 102 includes a housing container 106 and a removable cap 104 configured to be fastened onto the housing container 106. The housing container further includes an applicator body 110 configured to make contact with the skin of a user. The housing container 106 and the applicator body 110 may be made from a rigid polymer or a plastic material such as Polycarbonate (PC), Polymethyl methacrylate (PMMA), Polyvinyl chloride (PVC), Acrylonitrile Butadiene Styrene (ABS), High-density polyethylene (HDPE), Nylon or Polyamide (PA), Polytetrafluoroethylene (PTFE), Polypropylene (PP), and the like.

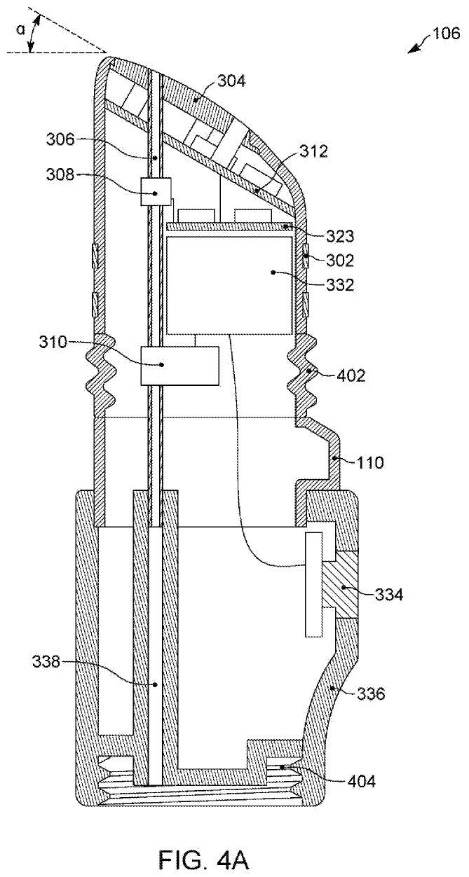

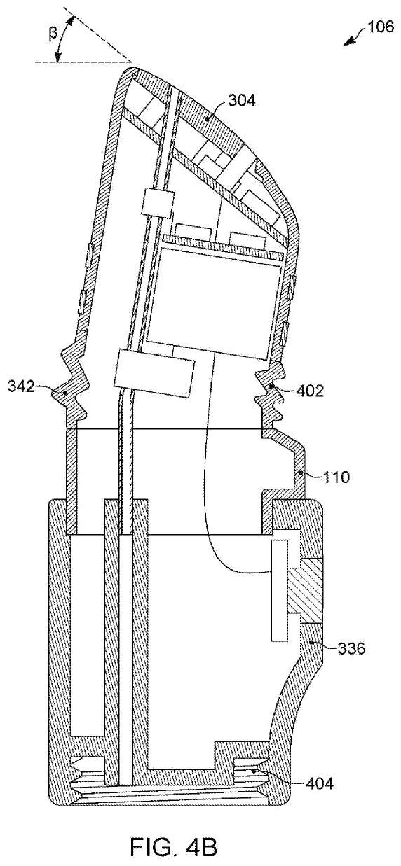

The serum container 108 is configured to store therewithin a serum formulated to be applied to the skin of the user. Furthermore, the housing container 106 may further include a first engaging arrangement (See FIGS. 4A and 4B) configured to engage with a second engaging arrangement 112 provided in an upper portion of the serum container 108. In that regard, the first and the second 112 engaging arrangements may be complementary to each other, such as mating internal-external thread, snap-fit arrangements, clips, tapered surfaces, and the like. The first and the second 112 engaging arrangements enable the fastening of the housing container 106 to the serum container 108.

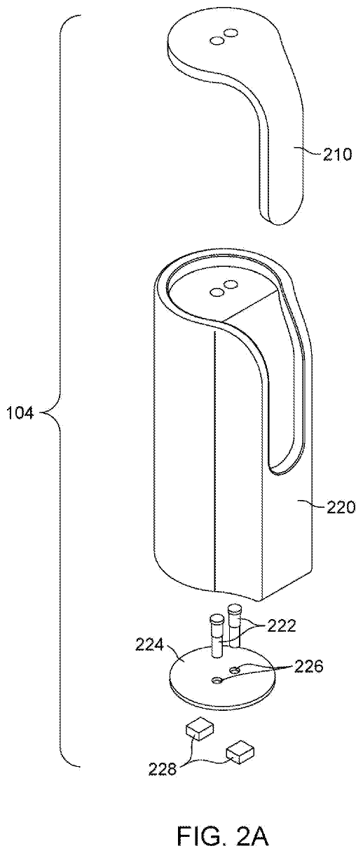

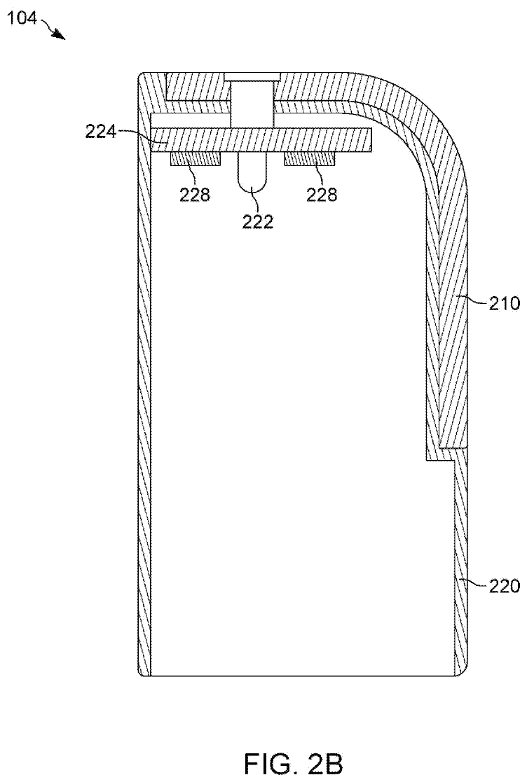

FIG. 2A illustrates an exploded view of the removable cap 104, in accordance with an embodiment of the present invention. FIG. 2B illustrates a sectional view of the removable cap 104, in accordance with an embodiment of the present invention. The removable cap 104 includes a power receiving terminal 210 configured to receive electrical power from an external electrical power source through a direct conducting connection or through electromagnetic induction. The removable cap 104 further includes a cap body 220 configured to enclose therewithin a cap Printed Circuit Board (PCB) 224. At least two power terminals 222 are electrically coupled to the cap PCB 224. The at least two power terminals 222 are configured to receive electrical power from the power receiving terminal 210. The at least two power terminals 222 protrude downwards from at least two respective apertures 226 provided in the cap PCB 224. Also, Ultraviolet (UV) light sources 228 are electrically coupled to the cap PCB 224 and are configured to emit UV light when activated with electrical current.

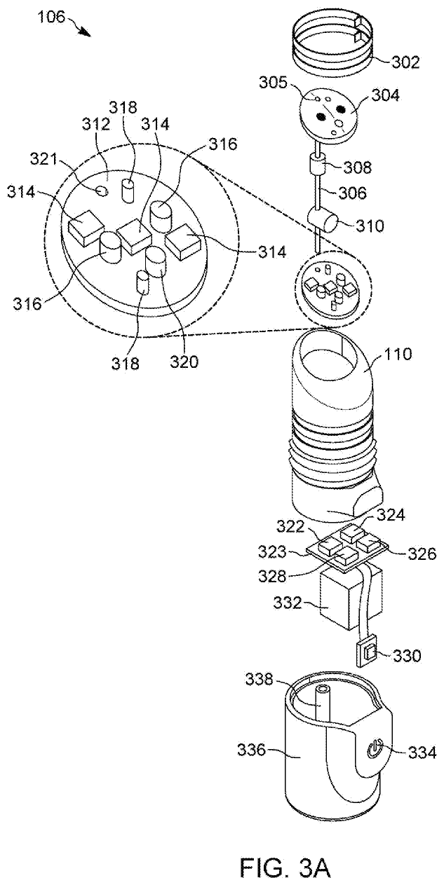

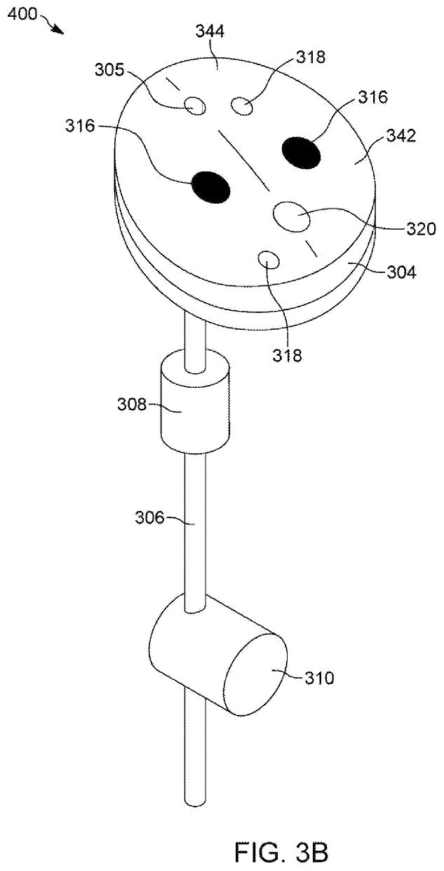

FIG. 3A illustrates an exploded view depicting the housing container 106 and elements contained within the housing container 106, in accordance with an embodiment of the present invention. The housing container 106 includes an applicator body 110. An applicator plate 304 has been provided in an upper portion of the applicator body 110. Furthermore, the applicator plate 304 is inclined at a predetermined angle of inclination (See FIGS. 4A and 4B). FIG. 3B illustrates the applicator plate 304 and the fluidic channel 306 coupled to the applicator plate 304, in accordance with an embodiment of the present invention. The applicator plate 304 has an upper surface 342. Furthermore, the applicator plate 304 includes a dispensing hole 305 located in an uphill portion 344 of the upper surface 342. The dispensing hole 305 is configured to be fluidically connected to the serum container 108 through a fluidic channel 306. In use, due to the location of the dispensing hole 305 on the uphill portion 344, the serum contained in the serum container 108 is configured to flow downhill from the dispensing hole 305 and along the inclined applicator plate 304. In several embodiments of the invention, the fluidic channel 306 may be provided with an electronically controlled valve 308 and an electronically controlled pump 310.

Referring to FIGS. 3A and 3B, a first housing PCB 312 is located underneath the applicator plate 304. One or more stimulation elements 314 are electrically coupled to the first housing PCB 312. In several embodiments of the invention, the one or more stimulation elements 314 may be selected from a group consisting of irradiation sources, heating elements, cooling elements, vibration elements, ultrasonic wave generators, electrodes, and combinations thereof. Furthermore, irradiation sources may be selected from a group consisting of Light Emitting Diodes (LEDs), and lasers. Additionally, the irradiation sources may be configured to operate in one or more of a pulse mode and a continuous mode. In the case of a stimulation element being an irradiation source, the stimulation element would be emitting electromagnetic radiation.

In the case of a stimulation element being a vibration element, the stimulation element would be a vibrating head connected to an eccentric mass rotating motor or a linear resonant motor. In case of a stimulation element being a heating element, the stimulation element may be selected from a group consisting of metal heating elements, ceramic heating elements, semiconductor heating elements, thick film heating elements, polymer-based heating elements, composite heating elements, and combination heating elements. In the case of a stimulation element being a cooling element, the stimulation element may be a thermoelectric cooler, also known as a Peltier heat pump. In the case of a stimulation element being an ultrasonic wave generator, a wave generation head may consist of a quartz crystal fused with a metal plate. The quartz crystal may produce ultrasonic waves due to the piezoelectric effect. Ultrasonic wave therapy may be used in applications such as treatment of chronic pain, improvement in blood circulation, and tissue repair.

In the case of a stimulation element being an electrode, the stimulation element may be embodied as an open-ended conductor. The electrode may then be able to provide Transcutaneous Electrical Nerve Stimulation (TENS), Electronic Muscle Stimulation (EMS), and Microcurrent Electrical Therapy (MET) to the inner surfaces of the oral cavity. TENS therapy uses low-voltage currents to provide pain relief. Electrical impulses are delivered through electrodes placed on the surface of the body of the user. The electrodes are placed at or near nerves where the pain is located or at certain known trigger points. EMS therapy is similar to TENS therapy, the difference being that EMS is applied to key muscle groups instead of a generalized application. The electrical signals in EMS cause certain muscles to undergo contractions and tightening. Moreover, electrical impulses in EMS are stronger when compared with TENS therapy. MET in contrast uses a current of amplitude less than 1 milliampere and a frequency of 0.5 Hz and is indicated for the treatment of pain.

Furthermore, it is envisaged that the applicator plate 304 may be modified in conformance with the one or more stimulation elements 314. For example, portions of the applicator plate 304 above the irradiation sources may be made diaphanous. Furthermore, portions of the applicator plate 304 above the electrodes may be made electrically conducting, above the heating and cooling elements may be made thermally conducting, above ultrasonic wave generators and vibratory elements may be made from material with high transmissibility.

The first housing PCB 312 further includes electrically coupled thereto at least two charging terminals 316. The at least two charging terminals 316 are flush with the upper surface 342 of the applicator plate 304. The at least two charging terminals 316 are configured to receive electrical power from the at least two respective power terminals 228 of the removable cap 104 and supply the received electrical power to a rechargeable electrical power source 332 located within the housing container 106 and below the applicator plate 304. The rechargeable electrical power source 332 may include rechargeable batteries such as Lithium-ion, Lithium-polymer, or Nickel-metal-hydride batteries and is configured to supply electrical power to the one or more stimulation elements 314 and other electronic components of the therapy applicator head 102. Furthermore, pressure sensors 318 are electrically coupled to the first housing PCB 312 and mechanically coupled to the applicator plate 304. The pressure sensors 318 are configured to sense the pressure applied onto the applicator plate 304. The pressure sensor 318 may be constructed as a sensing diaphragm, a piezoresistive transducer, a piezoelectric transducer, a capacitive transducer, a strain gauge, and the like.

Also, electrically connected to the first housing PCB 312 is a skin condition sensor 320 configured to generate skin condition data. In that regard, the skin condition sensor 320 may be mechanically coupled to the applicator plate 304 and be flush with the upper surface 342 of the applicator plate 304. The skin condition sensor 320 may be embodied as an optical sensor configured to capture images of the skin of the user. In several alternate embodiments of the invention, the skin condition sensor 320 may be displacement-based, a pressure sensor, or an accelerometer configured to generate signals due to abrupt changes in the surface of the skin, such as lesions, crusts, bumps, acne, etc. The first housing PCB 312 further includes a second aperture 321 configured to accommodate the fluidic channel 306.

The housing container 106 further includes a second housing PCB 323. The second housing PCB 323 includes, electrically connected thereto, a processor 322, a memory unit 324, a communication interface 326, and a storage device 328. During runtime, the memory unit 324 may include machine-readable instructions that may be executed by the processor 322. The first housing PCB 312, the second housing PCB 323, the rechargeable electrical power source 332, etc. may be fastened to the applicator body 110 using snap rings 302. Furthermore, a pressure switch 330 is electrically connected to the second housing PCB 323. The pressure switch 330 sits underneath a user interface 334 provided with a lower body 336 of the housing container 106. The user interface 334 may be embodied as a touch interface (resistive or capacitive type), a single control button, or a group of control toggles.

The user interface 334 is configured to receive control input signals to modify operational characteristics of the one or more stimulation elements 314. Some of the non-limiting examples of the operational characteristics may include wavelength, intensity, period of pulses for the irradiation sources, temperature and time duration of activation of heating and cooling elements, frequency or acceleration of vibratory elements, frequency, and intensity of sound waves of the ultrasonic wave generators, applied voltage and current at the electrodes, etc. The lower body 336 is configured to be coupled to the applicator body 110. Furthermore, the lower body 336 includes a channel housing 338 configured to accommodate the fluidic channel 306.

FIG. 4A illustrates a sectional view of the housing container 106 and the elements contained therewithin, in accordance with an embodiment of the present invention. The applicator plate 304 is inclined at a predetermined angle of inclination (α). However, the applicator body 110 has been provided with an inclination modifying mechanism 402 in the form of a corrugated or bellows-type surface. The corrugated or bellows-type surface of the inclination modifying mechanism 402 may be deformed to adjust the predetermined angle of inclination from (α) to (β) as illustrated in FIG. 4B. FIGS. 4A and 4B also depict the first engaging arrangement 404 of the housing container 106.

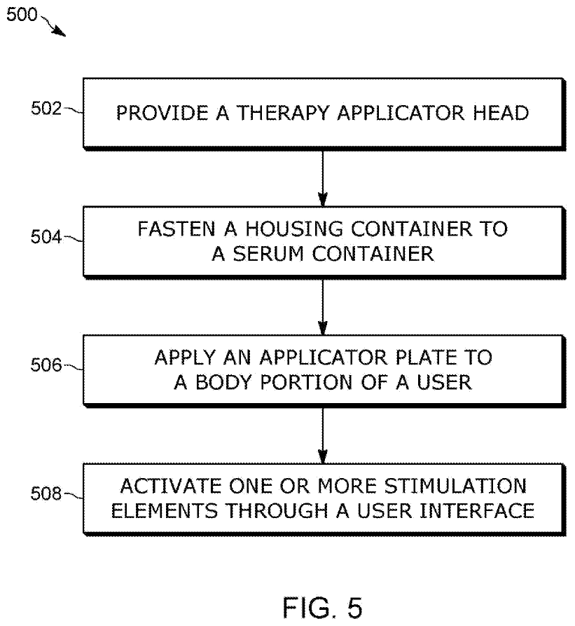

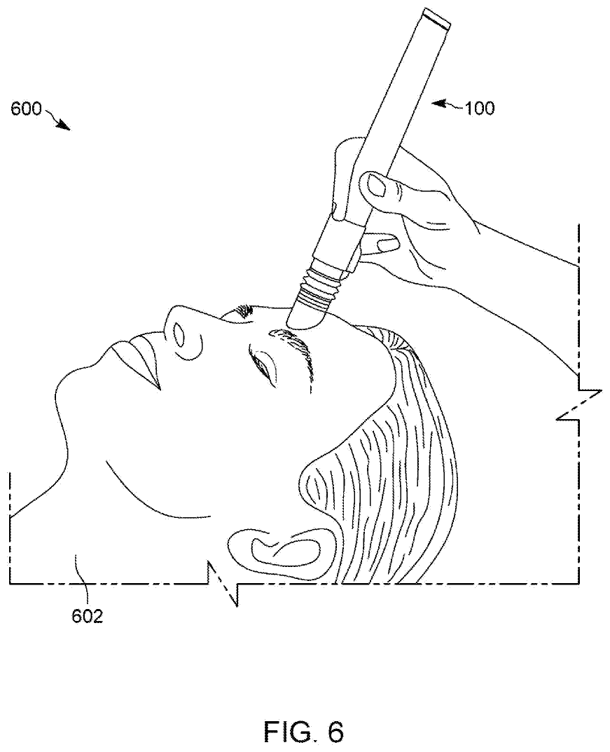

FIG. 5 illustrates a method 500 of using the therapy applicator head 102, in accordance with an embodiment of the present invention. The method 500 begins at Step 502 by providing the head 102 as disclosed in several embodiments of the discussion. At Step 504, the hosing container 106 including the applicator body 110 is fastened to the serum container 108 through the first engaging arrangement 404 of the housing container 106 and the second engaging arrangement 112 of the serum container 108. The dispensing hole 305, as a consequence, is fluidically connected to the serum container 108 through the fluidic channel 306. At Step 506, the applicator plate 304 is applied to a body portion of the user. FIG. 6 illustrates an application scenario 600 depicting the assembly 100 of the therapy applicator head 102 and the serum container 108 being applied to a facial region of user 602, in accordance with an embodiment of the present invention. As a result, the serum stored in the serum container 108 is applied, to the body portion of the user, through the fluidic channel 306.

In several embodiments of the invention, the processor 322 executing the machine-readable instructions, receives a pressure input from the pressure sensors 318 indicative of a predetermined amount of pressure applied to the applicator plate 304. Furthermore, the processor 322 then allows the serum to be dispensed from the dispensing hole 305. The predetermined amount of pressure would ensure that there is no accidental or unintended discharge of the serum from the dispensing hole 305. Furthermore, in several embodiments of the invention, the processor 322 may operate the electronically controlled valve 308 to allow the serum to be dispensed from the dispensing hole 305. In addition, or alternately, the processor 322 may operate the electronically controlled pump 310 to allow the serum to be dispensed from the dispensing hole 305.

At Step 508, the one or more stimulation elements 314 are activated by the user through the user interface 334. Furthermore, the user interface 334 receives control input signals to modify the operational characteristics of the one or more stimulation elements 314, the electronically controlled valve 308, and/or the electronically controlled pump 310. The processor 322 receives the control input signals from the user interface 334 and modifies the operational characteristics of the one or more stimulation elements 314, the electronically controlled valve 308, and/or the electronically controlled pump 310. In several embodiments of the invention, the processor 322 may further receive the skin condition data, such as the optical data, the displacement data, or the accelerometer data from the skin condition sensor 320. Furthermore, the processor 322 may then, in correlation with the skin condition data, further modify the operational characteristics of the one or more stimulation elements 314 and/or allow a predetermined amount of the serum to be dispensed from the dispensing hole 305.

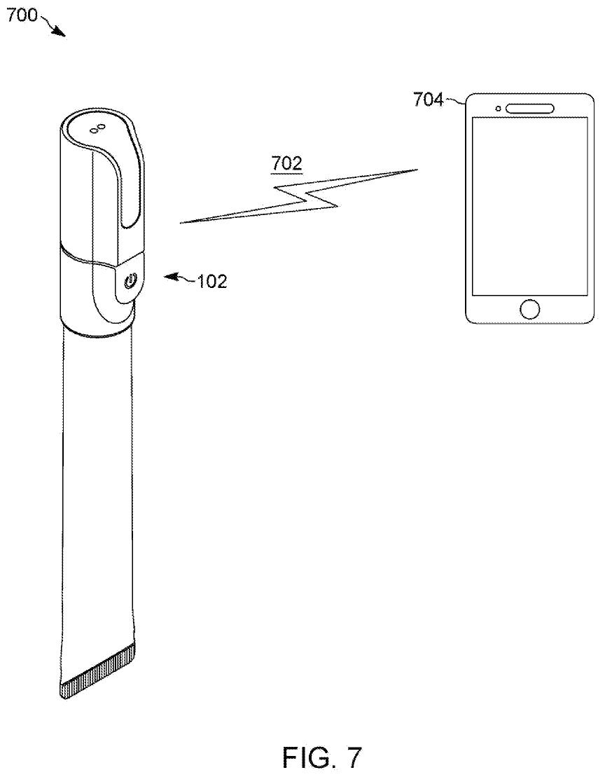

FIG. 7 illustrates an application scenario 700 depicting the therapy applicator head 102 in communication with a user computing device 704, in accordance with an embodiment of the present invention. The processor 322 may communicate with the user computing device 704 through the communication interface 326 and a communication network 702 and receive the control input signals for modifying the operational characteristics of the one or more stimulation elements 314, the electronically controlled valve 308, and/or the electronically controlled pump 310. The user computing device 704 in that regard, may be selected from a group consisting of smartphones, notebook PCs, tablet PCs, desktop PCs, and the like.

In several embodiments of the invention, after the use of the head 102, the removable cap 104 may be fastened onto the housing container 106. As a result, the at least two power terminals 222, enclosed within the removable cap 104, make contact with the at least two respective charging terminals 316 provided on the upper surface 342 of the applicator plate 304, to supply electrical power to the at least two charging terminals 316 from an external power source. Furthermore, the upper surface 342 is sterilized through the Ultraviolet (UV) light sources 228 included within the removable cap 104 and the one or more stimulation elements 314.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A therapy applicator head, the therapy applicator head comprising:
a housing container comprising an applicator body;
an applicator plate provided in an upper portion of the applicator body, the applicator plate inclined at a predetermined angle;
one or more stimulation elements located underneath the applicator plate; and
a rechargeable electrical power source located below the applicator plate, the rechargeable electrical power source configured to supply electrical power to the one or more stimulation elements,
wherein the applicator plate further comprises a dispensing hole located in an uphill portion on an upper surface of the applicator plate, the dispensing hole configured to be fluidically connected to a serum container through a fluidic channel, and
wherein, in use, a serum contained in the serum container is configured to flow downhill from the dispensing hole and along the inclined applicator plate.

2. The therapy applicator head as claimed in claim 1, wherein the applicator plate is made from a thermally conducting material and/or an electrically conducting material.

3. The therapy applicator head as claimed in claim 1, wherein the predetermined angle of the applicator plate is adjustable.

4. The therapy applicator head as claimed in claim 1, further comprising at least two charging terminals provided on the upper surface of the applicator plate, the at least two charging terminals configured to receive electrical power to charge the rechargeable electrical power source.

5. The therapy applicator head as claimed in claim 4, further comprising a removable cap configured to be fastened onto the housing container, wherein the removable cap comprises enclosed therewithin at least two power terminals configured to make contact with the at least two respective charging terminals, to supply electrical power to the at least two charging terminals from an external power source.

6. The therapy applicator head as claimed in claim 5, wherein the removable cap and the one or more stimulation elements comprise an Ultraviolet (UV) light source configured to sterilize the upper surface of the applicator plate.

7. The therapy applicator head as claimed in claim 1, wherein the housing container comprises a first engaging arrangement configured to engage with a second engaging arrangement of the serum container, the second engaging arrangement complementary to the first engaging arrangement, to fasten the housing container to the serum container.

8. The therapy applicator head as claimed in claim 1, wherein the one or more stimulation elements are selected from a group consisting of irradiation sources, heating elements, cooling elements, vibratory elements, ultrasonic wave generators, electrodes, and combinations thereof.

9. The therapy applicator head as claimed in claim 8, wherein the irradiation sources are configured to emit electromagnetic radiation in wavelengths ranging between 300 nm and 1200 nm.

10. The therapy applicator head as claimed in claim 1, further comprising a user interface configured to receive control input signals to modify operational characteristics of the one or more stimulation elements.

11. The therapy applicator head as claimed in claim 1, further comprising:
a processor;
a memory unit operably connected with a processor; and
a pressure sensor mechanically coupled to the applicator plate,
wherein the memory unit comprises machine-readable instructions, the machine-readable instructions when executed by the processor, enable the processor to:
receive a pressure input from the pressure sensor, indicative of a predetermined amount of pressure applied onto the applicator plate, and
allow the serum to be dispensed from the dispensing hole in response to sensing the predetermined amount of pressure.

12. The therapy applicator head as claimed in claim 11, wherein the processor is further enabled to operate an electronically controlled valve to allow the serum to be dispensed from the dispensing hole.

13. The therapy applicator head as claimed in claim 11, wherein the processor is further enabled to operate an electronically controlled pump to allow the serum to be dispensed from the dispensing hole.

14. The therapy applicator head as claimed in claim 11, further comprising a skin condition sensor mechanically coupled to the applicator plate, wherein the processor is further enabled to:
receive skin condition data from the skin condition sensor, and
in correlation with the skin condition data, perform one or more of:
modifying operational characteristics of the one or more stimulation elements, and
allowing a predetermined amount of serum to be dispensed from the dispensing hole.

15. A therapy applicator head, the therapy applicator head comprising:
a housing container comprising an applicator body;
an applicator plate provided in an upper portion of the applicator body;
one or more stimulation elements located underneath the applicator plate;
a rechargeable electrical power source located below the applicator plate, the rechargeable electrical power source configured to supply electrical power to the one or more stimulation elements;
at least two charging terminals provided on an upper surface of the applicator plate, the at least two charging terminals configured to receive electrical power to charge the rechargeable electrical power source; and
a removable cap configured to be fastened onto the housing container,
wherein the removable cap comprises enclosed therewithin at least two power terminals configured to make contact with the at least two respective charging terminals provided on the upper surface of the applicator plate, to supply electrical power to the at least two charging terminals from an external power source.

16. The therapy applicator head as claimed in claim 15, wherein the applicator plate inclined at a predetermined angle, the predetermined angle of the applicator plate being adjustable.

17. The therapy applicator head as claimed in claim 15, wherein the removable cap and the one or more stimulation elements comprise an Ultraviolet (UV) light source configured to sterilize the upper surface of the applicator plate.

18. A method of using a therapy applicator head, the method comprising:
providing the therapy applicator head, the therapy applicator head comprising:
a housing container comprising an applicator body,
an applicator plate provided in an upper portion of the applicator body, the applicator plate inclined at a predetermined angle,
one or more stimulation elements located underneath the applicator plate,
a rechargeable electrical power source located below the applicator plate, the rechargeable electrical power source configured to supply electrical power to the one or more stimulation elements, and
at least two charging terminals provided on an upper surface of the applicator plate, the at least two charging terminals configured to receive electrical power to charge the rechargeable electrical power source;
fastening the housing container to a serum container, the housing container comprising a first engaging arrangement configured to engage with a second engaging arrangement of the serum container, the second engaging arrangement complementary to the first engaging arrangement, wherein the applicator plate further comprises a dispensing hole on the upper surface of the applicator plate, the dispensing hole fluidically connected to the serum container through a fluidic channel;
applying the applicator plate to a body portion of a user, as a result wherein, a serum stored in the serum container is applied, to the body portion of the user, through the fluidic channel; and
activating the one or more stimulation elements through a user interface configured to receive control input signals to modify operational characteristics of the one or more stimulation elements.

19. The method as claimed in claim 18, further comprising fastening a removable cap onto the housing container, as a result wherein, at least two power terminals, enclosed within the removable cap, make contact with the at least two respective charging terminals provided on the upper surface of the applicator plate, to supply electrical power to the at least two charging terminals from an external power source.

20. The method as claimed in claim 18, further comprising sterilizing the upper surface of the applicator plate through an Ultraviolet (UV) light source comprised within the removable cap and the one or more stimulation elements.
